(19) United States
(12) Patent Application Publication
(10) Pub. No.: US 2022/0328131 A1
(43) Pub. Date: Oct. 13, 2022

(54) METHOD FOR SCREENING PATHOGENIC UNIPARENTAL DISOMY AND USE THEREOF

(71) Applicants: Guangzhou Kingmed Center for Clinical Laboratory, Guangzhou City (CN); Guangzhou KingMed Diagnostics Group Co., Ltd., Guangzhou, Guangdong (CN)

(72) Inventors: Jingxing LIU, Guangzhou, Guangdong (CN); Weiwei ZHAO, Guangzhou, Guangdong (CN); Baixue CHEN, Guangzhou, Guangdong (CN); Shihui YU, Guangzhou, Guangdong (CN); Changshun YU, Guangzhou, Guangdong (CN); Lina XIANG, Guangzhou, Guangdong (CN)

(21) Appl. No.: 17/616,714

(22) PCT Filed: Jun. 3, 2020
(86) PCT No.: PCT/CN2020/094125

§ 371 Date: Dec. 6, 2021

(30) Foreign Application Priority Data
Jun. 6, 2019 (CN) 201910491767.1

(51) Int. Cl. G16B 20/20; G16H 10/40; G16H 50/70

(52) U.S. Cl. CPC G16B 20/20; G16H 10/40; G16H 50/70

(57) ABSTRACT

A method of screening a pathogenic uniparental disomy and a use thereof is provided. The method includes the steps as follows: obtaining data: obtaining whole exome sequencing data; screening for sites: screening and obtaining mutations under pre-determined conditions; judging LOH: performing LOH judgement according to the mutations obtained above; and judging UPD: judging UPD according to the LOH judgement, wherein when an amount of chromosomes with LOH exceeds 2, a sample is judged as a consanguineous marriage; when there is a single copy of a region with LOH, a sample is judged as a fragment deletion; and other samples are judged as UPD when there are regions with LOH. In the method, specific mutated sites are screened out to perform LOH judgment, to finally obtain the results for UPD judgment. The method is based on the whole exome sequencing data, indicating the risk of pathogenic UPD alongside conventional screening of pathogenic mutations, without additional experiments and labor cost.

### CROSS-REFERENCE TO RELATED APPLICATIONS

This application claims priority to PCT Application No. PCT/CN2020/094125, having a filing date of Jun. 3, 2020, which is based on Chinese Application No. 201910491767.1, having a filing date of Jun. 6, 2019, the entire contents both of which are hereby incorporated by reference.

### FIELD OF TECHNOLOGY

The following relates to the technical field of genetic detection, particularly, it relates to a method for screening a pathogenic uniparental disomy and a use thereof.

### BACKGROUND

Genomic imprinting, also known as genetic imprinting, is a genetic process where one gene or genomic region is marked in accordance to its parent of origin through a biochemical approach. The gene is named as an imprinted gene whose expression depends on the origin (paternal line and maternal line) of chromosome which the gene is located in and depends on whether the gene is silenced (the silencing mechanism is mostly methylation) on the chromosome from which it is originated. Some imprinted genes are only expressed in maternal chromosomes, while some others are expressed in paternal chromosomes.

In a normal diplont, one chromosome of each homologous pair comes from the father and one comes from the mother. UniParental Disomy (UPD for short) refers to a situation where a pair of homologous chromosomes (or some regions on the chromosome) come from only one parent. If such regions include imprinted genes, they may result in disordered expression of the genes.

At present, the method for diagnosing UPD mainly includes a methylation level detection method or a SNP chip-based method. Specifically, the methylation level detection method is to detect whether the methylation levels of the same regions on a pair of homologous chromosomes are the same. However, the methylation method can only deal with small regions on a part of chromosomes, and different experiments are required to be designed for different regions, which results in low efficiency and slow speed. Thus, the methylation method is not suitable for a genome-wide screening. As for the SNP chip-based method, it is to detect whether there are large contiguous homozygous regions by using a SNP chip, and has the disadvantage of high cost, and its targeted probes comprise polymorphism sites, so pathogenic micro-mutations (point mutations, small insertions/deletions) can not be detected at the same time.

### SUMMARY

An aspect relates to a method for screening a pathogenic uniparental disomy, which can be used in a device for screening, according to whole exome sequencing data, to check conventional pathogenic mutations, and meanwhile, to indicate a risk of pathogenic UPD, without additional experiments and labor costs.

A method for screening a pathogenic uniparental disomy comprises the steps as follows:

obtaining data: obtaining whole exome sequencing data;

screening for sites: screening and obtaining mutations under pre-determined conditions;

judging LOH: performing LOH (loss of heterozygosity) judgement according to the mutations obtained above; and a region is judged to be LOH when a product of an amount of contiguous homozygous sites and their coverage range is greater than a pre-set value; and

judging UPD: judging UPD according to the LOH judgement, wherein when an amount of chromosomes with LOH exceeds 2, a sample is judged as a consanguineous marriage; when there is a single copy of a region with LOH, a sample is judged as a fragment deletion; and other samples are judged as UPD when there are regions with LOH.

The whole exome sequencing is currently the most common method for detecting genetic defect disease. It can be used for detecting pathogenic mutations, small insertions/deletions, copy number variants, etc., and therefore is a first option for most patients suffering from pathogenic mutations, small insertions/deletions, copy number variants. An additional step of screening pathogenic UPD based on the whole exome sequencing can improve the positive diagnosis rate without increasing any cost.

Considering that UPD inherits from two copies of the same chromosome of one parent, it appears that all bases in the region are homozygous, i.e., loss of heterozygosity (LOH). There are three main conditions resulting in LOH, i.e. fragment deletion, UPD and consanguineous marriage. The LOH caused by these three conditions is different in fragment size, distribution and clinical manifestations such that it is possible to judge whether UPD exists by detecting LOH. Embodiments have screened out specific mutated sites to perform LOH judgment, and finally to obtain a judgment result of UPD.

For the judgment of consanguineous marriage, as UPD occurs occasionally and has a very low probability to occur on multiple chromosomes at the same time, samples in a consanguineous marriage can be distinguished out. That is, when the amount of chromosomes with LOH exceeds 2 (i.e., more than 2), a sample is judged as consanguineous marriage. For the judgment of fragment deletion, samples can be judged according to conventional methods. For example, samples can be judged in combination with the copy number variation (CNV) analysis result of whole exome sequencing. That is, the depth-of-coverage of sequencing data of the LOH region is compared with that of other samples in the same batch. If the CNV analysis indicates that the LOH region is a single copy, a sample is judged as a fragment deletion; in particular, deleting a large region is generally fatal, thus, if the LOH region is more than half of the entire chromosome, even the entire chromosome and the sample is not from an embryo, it can be basically excluded that the sample is judged as fragment deletion.

In one example, the mutations under pre-determined conditions are screened and obtained through the following approaches:

screening for high-quality mutation sites: screening for high-quality mutation sites from the whole exome sequencing data;

removing Y chromosome mutations: removing Y chromosome mutations from the above mutation sites;

screening for point mutations: screening for point mutations from the mutations obtained in the step of removing Y chromosome mutations;

screening for allele frequency: screening for sites which are located in the point mutations in the previous step to obtain the sites which have a population allele frequency of less than 0.7 in each race in a population database; and

screening for mutation frequency: removing sites which have a mutation frequency of heterozygous sites of higher than 70%, and removing sites which have a mutation frequency of homozygous sites of less than 85% from the sites which are located in point mutations in the previous step, thereby obtaining the mutations under predetermined conditions.

In one example, in the step of screening for high-quality mutation sites, the high-quality mutation sites are those passed through a quality control of GATK-VQSR, and having a total coverage range of more than 40X and a mutation frequency of greater than 30%.

In one example, a step of excluding false positive sites is further included between the step of screening for allele frequency and the step of screening for mutation frequency, wherein the step of excluding false positive sites is performed according to the Hardy-Weinberg balance, by excluding false positive sites from a frequency database in a regional population to be evaluated.

In one example, the step of screening for high-quality mutation sites further includes a step of quality control, wherein the step of quality control is used to detect the amount of mutations obtained by the screening. If the amount of mutations is greater than or equal to 10,000, the step of quality control indicates PASS; if the amount of mutations is less than 10,000, the step of quality control indicates FAIL.

In one example, in the step of judging LOH, the amount of contiguous homozygous sites is greater than or equal to 20, and their coverage range is greater than or equal to 3 Mbp.

In one example, in the step of judging LOH, if the product of the amount of contiguous homozygous sites and their coverage range is greater than 200 Mbp, a region is judged to be LOH.

In one example, the step of judging UPD further includes a step of judging a pathogenic risk. In the step of judging a pathogenic risk, the LOH region which is judged to be UPD is further compared with imprinted genes. When the LOH region does not cover an imprinted gene or a corresponding band, a sample is indicated as a benign UPD; when the LOH region covers the imprinted gene or the corresponding band, a sample is indicated as being at risk of pathogenic UPD.

The present disclosure also provides a use of the above-mentioned method for screening a pathogenic uniparental disomy in preparation of a device for screening a pathogenic uniparental disomy.

It is another aspect to provide a device for screening a pathogenic uniparental disomy, comprising:

a module of data acquisition, configured for obtaining whole exome sequencing data;

a module of site screening, configured for screening for mutations under pre-determined conditions;

a module of LOH judgment, configured for performing LOH judgment according to the mutations obtained above; and a region is judged to be LOH when a product of an amount of contiguous homozygous sites and their coverage range is greater than a pre-set value; and

a module of UPD judgment, configured for performing UPD judgement according to the LOH judgment, wherein when an amount of chromosomes with LOH exceeds 2, a sample is judged as a consanguineous marriage; when there is a single copy of a region with LOH, a sample is judged as a fragment deletion; and other samples are judged as UPD when there are regions with LOH.

The whole exome sequencing is a common method for detecting genetic defect disease at present, and it can be used for detecting pathogenic mutations, small insertions/deletions, copy number variants, etc., and therefore is a first option for most patients suffering from pathogenic mutations, small insertions/deletions, copy number variants. An additional step of screening pathogenic UPD based on the whole exome sequencing can improve the positive diagnosis rate without increasing any cost.

Considering that UPD inherits from two copies of the same chromosome of one parent, it appears that all bases in the region are homozygous, i.e., loss of heterozygosity (LOH). There are three main conditions resulting in LOH, i.e., fragment deletion, UPD and consanguineous marriage. The LOH caused by these three conditions is different in fragment size, distribution and clinical manifestations such that it is possible to judge whether UPD exists, by detecting LOH. Embodiments can screen out specific mutation sites to perform LOH judgment, and finally to obtain a judgment result of UPD.

For the judgment of consanguineous marriage, as UPD occurs occasionally and has a very low probability to occur on multiple chromosomes at the same time, samples in a consanguineous marriage can be distinguished out. That is, when the amount of chromosomes with LOH exceeds 2 (i.e., more than 2), a sample is judged as consanguineous marriage. For the judgment of fragment deletion, samples can be judged according to conventional methods. For example, samples can be judged in combination with the copy number variation (CNV) analysis result of whole exome sequencing. That is, the depth-of-coverage of sequencing data of the LOH region is compared with that of other samples in the same batch. If the CNV analysis indicates that the LOH region is a single copy, a sample is judged as a fragment deletion; in particular, deleting a large region is generally fatal, thus, if the LOH region is more than half of the entire chromosome, even the entire chromosome and the sample is not from an embryo, it can be basically excluded that the sample is judged as fragment deletions.

In one example, the mutations under pre-determined conditions are screened and obtained through the following approaches:

screening for high-quality mutation sites: screening for high-quality mutation sites from whole exome sequencing data;

removing Y chromosome mutations: removing Y chromosome mutations from the above mutation sites;

screening for point mutations: screening for point mutations from the mutations obtained in the step of removing Y chromosome mutations;

screening for allele frequency: screening for sites which are located in the point mutations in the previous step to obtain sites which have a population allele frequency of less than 0.7 in each race in a population database; and

screening for mutation frequency: removing sites which have a mutation frequency of heterozygous sites of higher than 70%, and removing sites which have a mutation frequency of homozygous sites of less than 85% from the sites which are located in point mutations in the previous step, thereby obtaining the mutations under predetermined conditions.

An analysis of the above-mentioned mutations can eliminate the impacts on a LOH judgement from false positive mutations, somatic mutations, and high-frequent mutations in the population, so that the judgment is accurate. For example, as a large LOH region, including some false positive mutations or somatic heterozygous mutations inside, is split into small LOH fragments, when each of the small LOH fragments cannot reach a pre-set length threshold (such as 3 M), the region will become unidentifiable, resulting in an uncertain judgment.

The above-mentioned population database includes 1000 Genomes, ESP6500, ExAC, goma AD, etc., and the race can be classified into East Asians, South Asians, African/African American, American, Finnish, non-Finnish European, etc.

In one example, in the module of screening for high-quality mutation sites, the high-quality mutation sites are those passed through a quality control of GATK-VQSR, and having a total coverage range of more than 40X and a mutation frequency of greater than 30%.

The “passed through a quality control of GATK-VQSR” as mentioned above means that the result of variant quality score recalibration obtained in GATK software is PASS; “total coverage range of more than 40X” means that more than 40 effective “reads” are covered at this site. The above-mentioned “mutation frequency of greater than 30%” refers to a proportion of “reads” for sites comprising mutated bases to all “reads”.

In one example, a module of excluding false positive site is further included between the module of allele frequency screening and the module of mutation frequency screening, wherein the module of excluding false positive site is performed according to the Hardy-Weinberg balance, by excluding false positive sites from a frequency database in a regional population to be evaluated. The “frequency database in a regional population to be evaluated” refers to a frequency database in the region where a subject to be evaluated is located. That is to say, false positive sites are excluded on the basis of conditions of such region.

In one example, the module of site screening further includes a quality control unit, wherein the quality control unit is used to detect the amount of mutations obtained by the screening. If the amount of mutations is greater than or equal to 10,000, the quality control unit indicates PASS; if the amount of mutations is less than 10,000, the of quality control unit indicates FAIL. If the amount of mutations is insufficient, the amount of contiguous homozygous sites is not enough, resulting in that there is no statistical significance.

In one example, in the module of LOH judgment, the amount of contiguous homozygous sites is greater than or equal to 20, and the coverage range is greater than or equal to 3 Mbp.

In one example, in the module of LOH judgment, if the product of the amount of contiguous homozygous sites and the coverage range thereof is greater than 200 Mbp, a region is judged to be LOH. For example, the coverage range of contiguous homozygous (Hom) sites is 5 Mbp, and the amount of Hom sites is 60, 60×5>200, and therefore the region is judged to be LOH.

The above pre-set value, i.e., 200 Mbp, is a threshold value obtained through repeated experiments and constant tests, and it has the advantages of accurate judgment and low misjudgment rate.

In one example, the module of UPD judgment further includes a unit of judging a pathogenic risk. In the unit of judging a pathogenic risk, the LOH region which is judged to be UPD is further compared with an imprinted gene. When the LOH region does not cover an imprinted gene or a corresponding band, this region is judged as a benign UPD; if the LOH region covers the imprinted gene or the corresponding band, the region is judged as being at risk of pathogenic UPD.

It is another aspect to provide a storage medium, comprising a stored program which achieves functions of the above-mentioned modules.

It is another aspect to provide a processor, which is used for running a program that realizes the functions of the above-mentioned modules.

Compared with the conventional art, the present disclosure has the benefits as follows:

The method for screening a pathogenic uniparental disomy of the present disclosure is analyzed and judged by successively performing data acquisition, sites screening, LOH and UPD judgements. The specific mutated sites are screened out, followed by performing LOH judgement, to finally obtain a result for UPD judgement. The method is based on the whole exome sequencing data, indicating the risk of pathogenic UPD alongside conventional screening of pathogenic mutations, without additional experiments and labor cost.

### BRIEF DESCRIPTION

Some of examples will be described in detail, with reference to the following figures, wherein like designations denote like members, wherein:

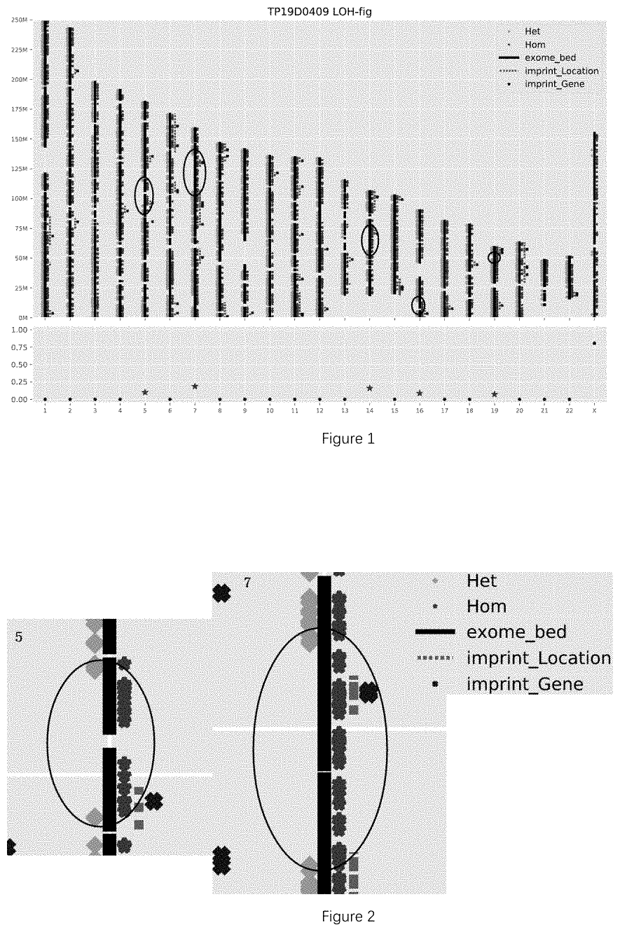

FIG. 1 shows a diagram of LOH distribution on chromosomes in Example 1;

FIG. 2 shows an enlarged diagram of LOH distributed on chromosomes 5 and 7 of FIG. 1;

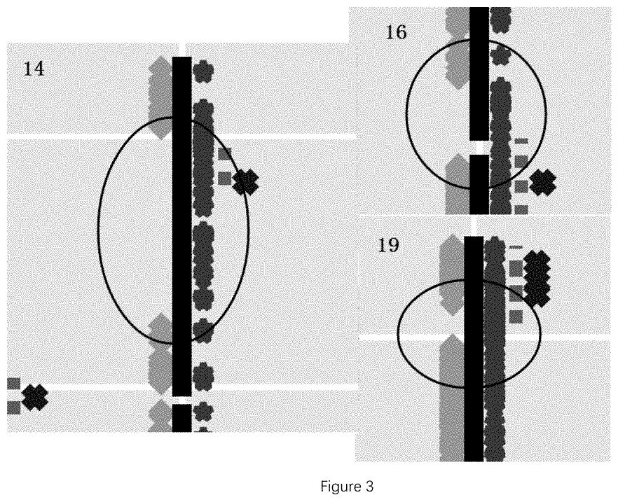

FIG. 3 shows an enlarged diagram of LOH distributed on chromosome 14, 16 and 19 of FIG. 1;

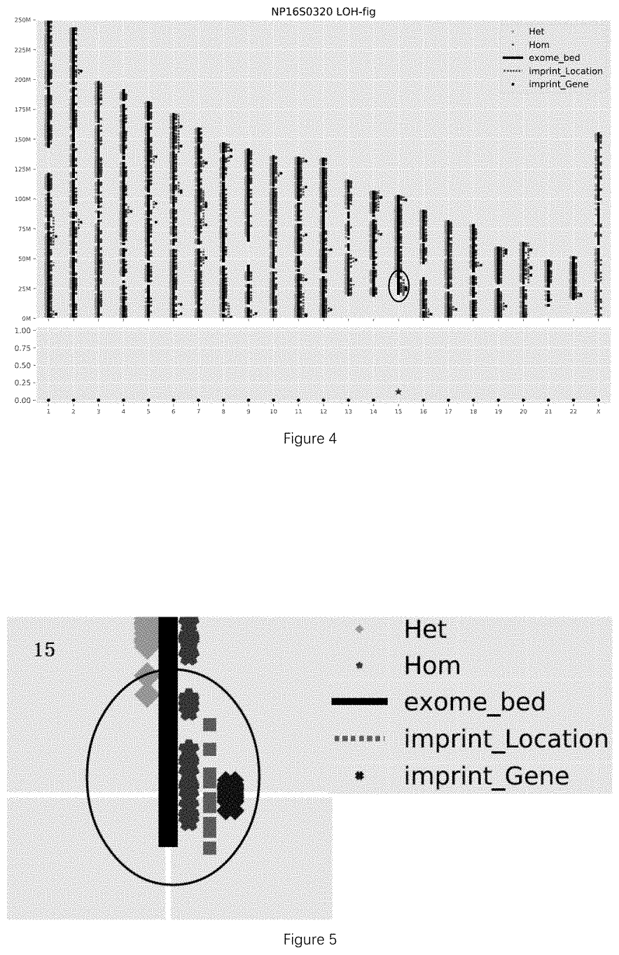

FIG. 4 shows a distribution diagram of LOH on chromosomes in Example 2;

FIG. 5 shows an enlarged diagram of LOH distributed on chromosome 15 of FIG. 4;

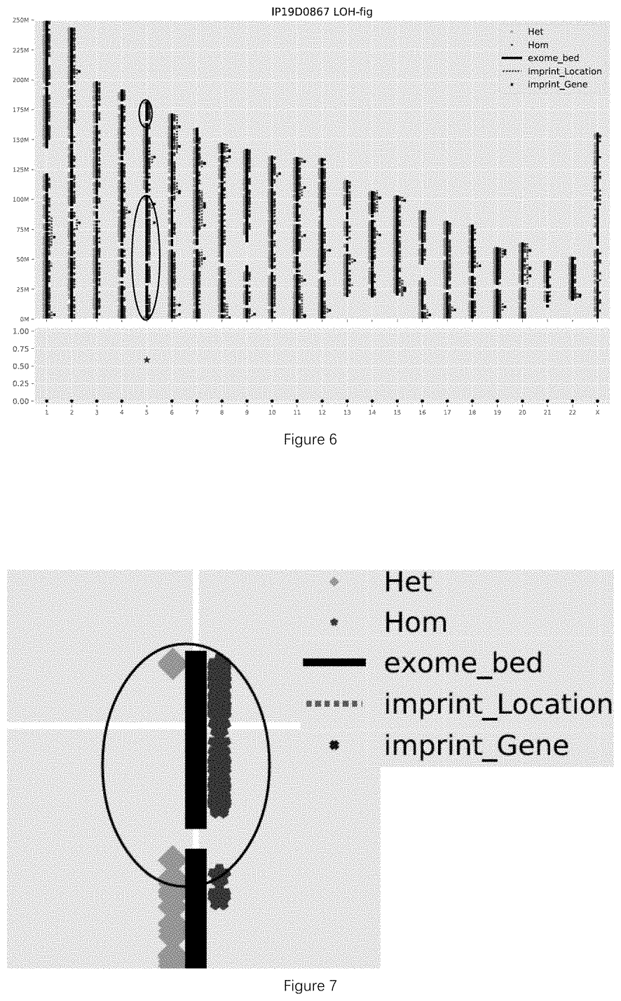

FIG. 6 shows a distribution diagram of LOH on chromosomes in Example 3;

FIG. 7 shows an enlarged diagram of LOH 12.57 (M) distributed on chromosome 5 of FIG. 6;

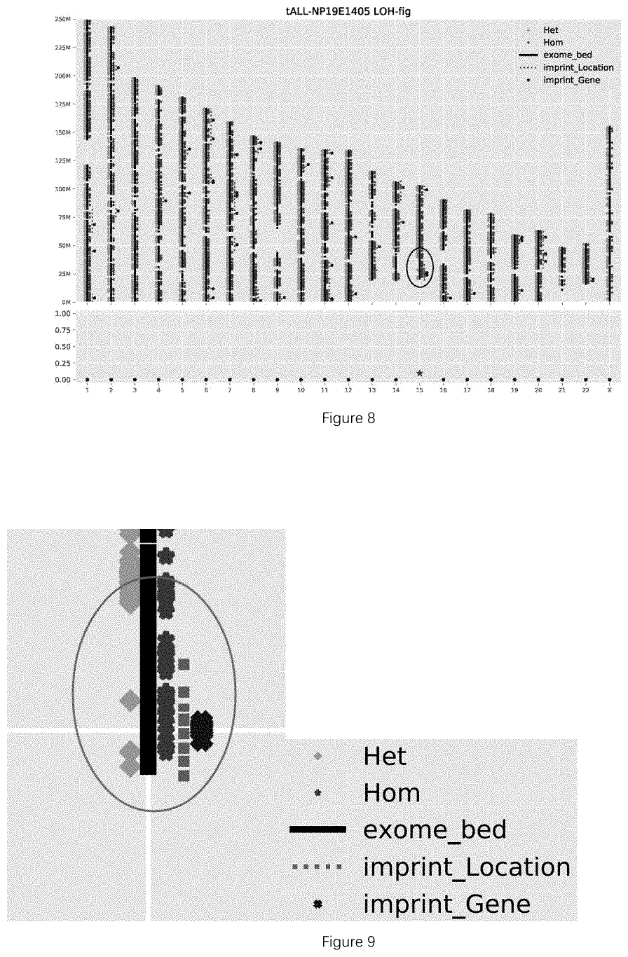

FIG. 8 shows a distribution diagram of LOH on chromosomes of sample NP19E1405 in Example 5;

FIG. 9 shows an enlarged diagram of LOH distributed on chromosome 15 of FIG. 8;

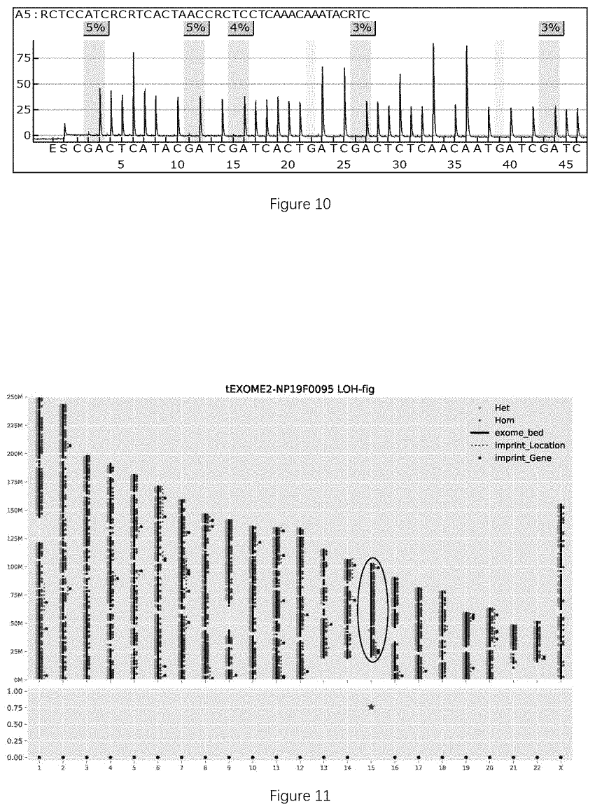

FIG. 10 shows a schematic diagram indicating the verified results of sample NP19E1405 in the methylation test;

FIG. 11 shows a distribution diagram of LOH on chromosomes of sample NP19F0095 in Example 5;

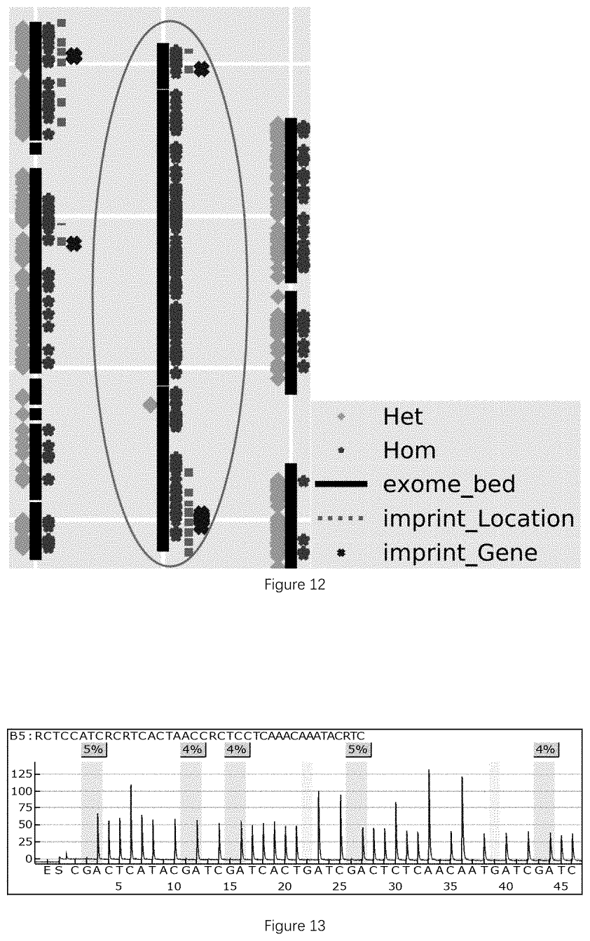

FIG. 12 shows an enlarged diagram of LOH distributed on chromosome 15 of FIG. 11;

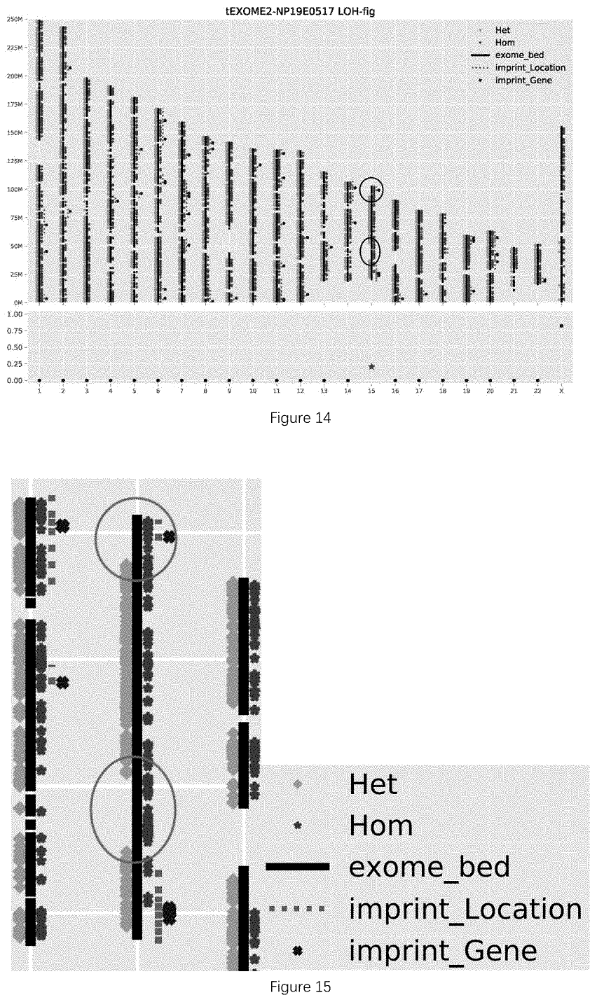

FIG. 13 shows a schematic diagram indicating the verified results of sample NP19F0095 in the methylation test;

FIG. 14 shows a distribution diagram of LOH on chromosomes of sample NP19E0517 in Example 5;

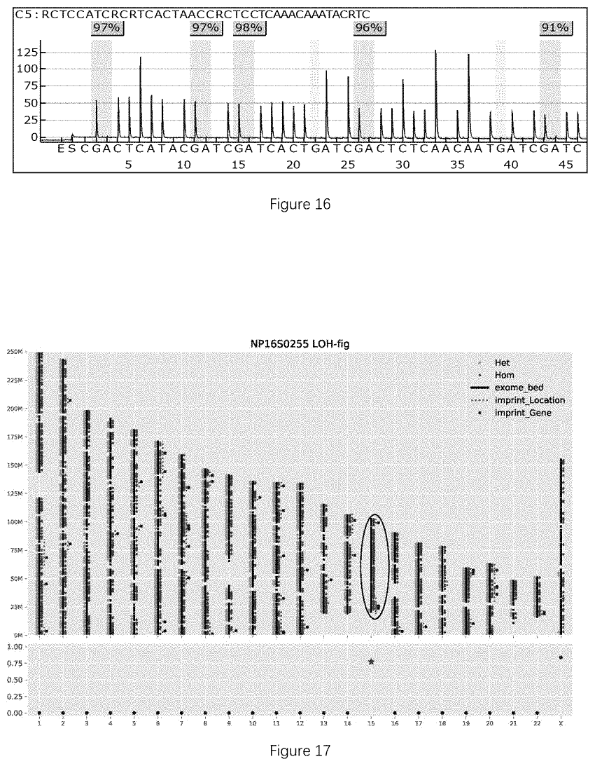

FIG. 15 shows an enlarged diagram of LOH distributed on chromosome 15 of FIG. 14;

FIG. 16 shows a schematic diagram indicating the verified results of sample NP19E0517 in the methylation test;

FIG. 17 shows a distribution diagram of LOH on chromosomes of sample NP16S0255 in Example 5;

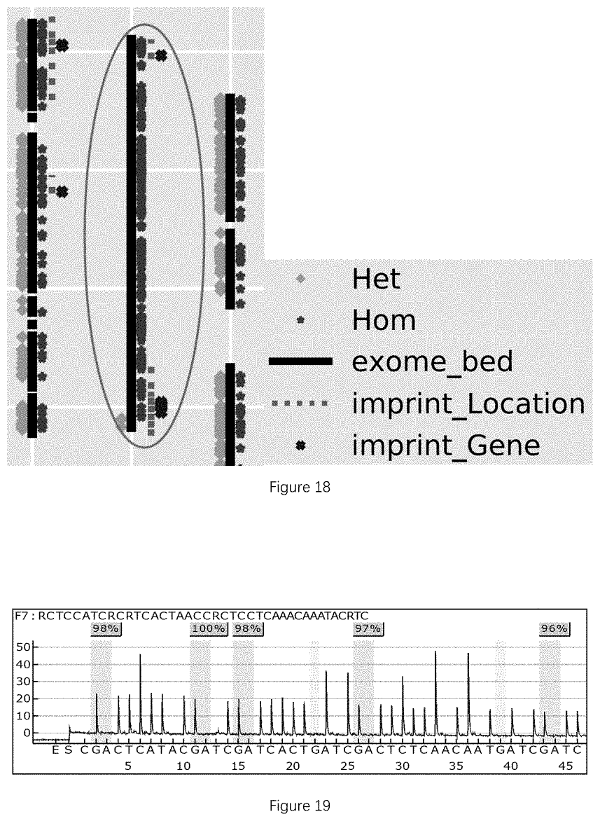

FIG. 18 shows an enlarged diagram of LOH distributed on chromosome 15 of FIG. 17;

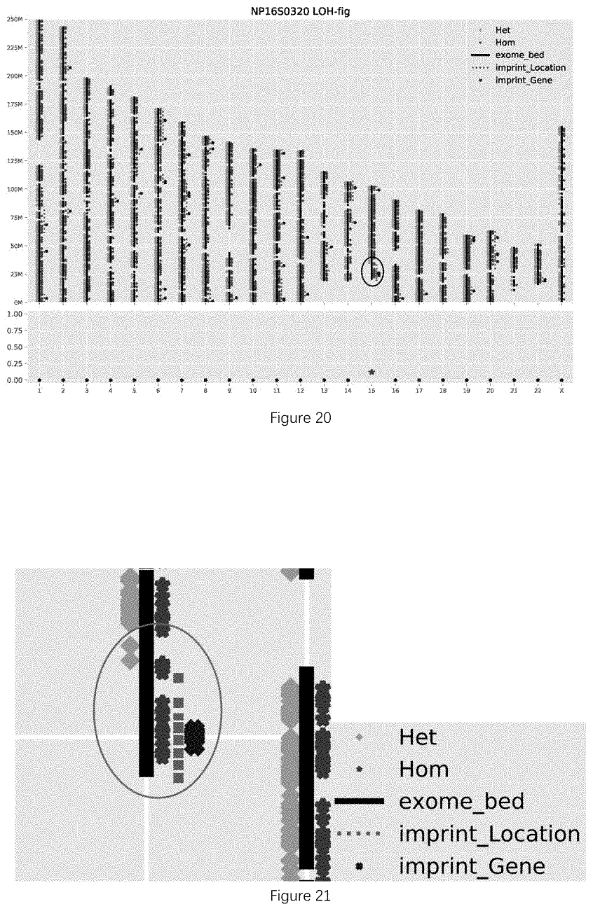

FIG. 19 shows a schematic diagram indicating the verified results of sample NP16S0255 in the methylation test;

FIG. 20 shows a distribution diagram of LOH on chromosomes of sample NP16S0320 in Example 5;

FIG. 21 shows an enlarged diagram of LOH distributed on chromosome 15 of FIG. 20; and

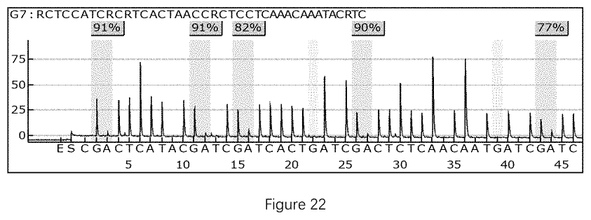

FIG. 22 shows a schematic diagram indicating the verified results of sample NP16S0320 in the methylation test;

wherein, in FIGS. 1, 4, 6, 8, 11, 14, 17, and 20, the abscissa indicates a serial number of each chromosome, the lower half of the figures shows proportions of lengths of contiguous homozygous sites to the entire chromosome, while the upper half of the figures shows the distribution of mutation sites on each chromosome; and

in the FIGS. 2, 3, 5, 7, 9, 12, 15, 18, and 21 showing enlarged diagrams of LOHs, the black line in the middle is an exome bed, the diamond points on the left is detected heterozygous (Het) mutations, and the five-pointed star points on the right is detected homozygous (Hom) mutations, the dotted line on the right is an imprinted location, and the cross points on the imprinted location are imprinted genes.

### DETAILED DESCRIPTION

For better understanding of the present disclosure, the present disclosure will be fully described below with reference to the relevant accompanying figures. The preferred embodiments are shown in the figures. However, the present disclosure can be implemented in many different forms and is not limited to the embodiments described herein. Rather, these embodiments are provided for the purpose of making the disclosed contents of the present disclosure more thorough and complete.

Unless otherwise defined, all technical and scientific terms used herein have the same meaning as those normally understood by one skilled in the art in the technical field belonging to the present disclosure. The terms used in the description of the present disclosure herein are only for the purpose of describing embodiments, and are not intended to limit the present disclosure. The term “and/or” used herein comprises anyone or all combinations of one or more corresponding items listed herein.

### EXAMPLE 1

A method for screening a pathogenic uniparental disomy, comprise the steps as follows:

1. Obtaining Data

The whole exome sequencing data of one sample was obtained, wherein there were 59312 mutations.

2. Screening for Sites

2.1 Screening for High-quality Mutation Sites

The high-quality mutation sites were screened in the whole exome sequencing data, specifically, the high-quality mutation sites were those passed through a quality control of GATK-VQSR, and having a total coverage range of more than 40X and a mutation frequency of greater than 30%. In this sample, there were 45260 mutations.

2.2 Removing Y Chromosome Mutations

The mutations on Y chromosome were removed from the above mutation sites, to obtain 45256 mutations.

2.3 Screening for Point Mutations

The point mutations were screened out from the mutations obtained in the step of removing Y chromosome to obtain 41273 mutations.

2.4 Screening for Allele Frequency

Sites which had a population allele frequencies of less than 0.7 in each race (East Asians, South Asians, African/African American, American, Finnish, non-Finnish European) in the population database (1000 Genomes, ESP6500, ExAC, gnomAD) were screened out from the point mutations obtained in the previous step, thereby obtaining 22,231 mutations.

2.5 Excluding False Positive Sites

According to the Hardy-Weinberg balance, the false positive sites were excluded from a frequency database in a regional population to be evaluated thereby obtaining 21,705 mutations.

2.6 Screening for Mutation Frequency

Sites which had a mutation frequency of heterozygous sites of higher than 70% and sites which had a mutation frequency of homozygous sites of less than 85% were removed from the above-mentioned point mutations in the previous step, thereby obtaining 21644 mutations under pre-determined conditions.

3. Judging LOH

For the above-mentioned sites, a region was judged to be LOH, if a product of an amount of contiguous homozygous sites and the coverage range thereof was greater than 200 Mbp, wherein the amount of contiguous homozygous sites was greater than or equal to 20, and the coverage range was greater than or equal to 3 Mbp.

According to the above rule, there were 5 LOH regions detected among the sample of Example 1, as shown in TABLE 1.

TABLE 1
LOH Regions

| | | | | Coverage | | |
|---|---|---|---|---|---|---|
| | | | | Range of | | |
| | | | Amount of | contiguous | | |
| | Start | End | homozygous | homozygous | Imprinted | Imprinted |
| Chromosome | Position | Position | mutations | sites (M) | gene | band |
| chr5 | 94860194 | 112927900 | 50 | 18.07 | ERAP2 | 5q15 |
| chr7 | 105752555 | 135329690 | 99 | 29.58 | MEST, | 7q21-q22, |
| | | | | | KLF14, | 7q22, 7q32, |
| | | | | | CPA4, | 7q32.2, |
| | | | | | MESTIT1 | 7q32.3 |
| chr14 | 58563694 | 75747258 | 88 | 17.18 | SMOC1 | 14q24.2 |
| chr16 | 5132636 | 13003248 | 45 | 7.87 | | 16p13.3 |
| chr19 | 49096065 | 53345414 | 98 | 4.25 | | 19q13.4 |

It can be seen from the above results that five LOH regions are located on five chromosomes, respectively. FIG. 1 shows a distribution diagram of the five LOH regions on the chromosomes, wherein the ellipse represents LOH regions. FIGS. 2 and 3 show enlarged diagram of LOH distribution on chromosomes 5, 7, 14, 16, and 19, respectively.

4. Judging UPD

As the five LOH regions were located on five chromosomes, respectively, the sample was judged as consanguineous marriage, rather than UPD. pathogenicity.

The sample is proved to be offspring of consanguineous marriage later.

### EXAMPLE 2

A screening of a pathogenic UPD was performed on a sample by using the method of Example 1, wherein:

1. Obtaining Data

It was performed with reference to Example 1.

2. Screening for Sites

It was performed with reference to Example 1, and 22210 mutations meeting the pre-determined conditions were obtained.

3. Judging LOH

For the above obtained sites, a region was judged to be LOH if a product of an amount of contiguous homozygous sites and the coverage range thereof was greater than 200 Mbp, wherein the amount of contiguous homozygous sites was greater than or equal to 20, and the coverage range was greater than or equal to 3 Mbp.

According to the above rule, there was 1 LOH region detected in the sample of this example, as shown in TABLE 2.

TABLE 2
LOH Regions

| | | | | Coverage | | |
|---|---|---|---|---|---|---|
| | | | | Range of | | |
| | | | Amount of | contiguous | | |
| | Start | End | homozygous | homozygous | | Imprinted |
| Chromosome | Position | Position | mutations | sites(M) | Imprinted gene | band |
| chr15 | 22369343 | 34649247 | 19 | 12.28 | SNRPN, MAGEL2, | 15q11-q12, |
| | | | | | NDN, SNORD107, | 15q11-q13, |
| | | | | | SNORD108, SNORD109A, | 15q11.2, |
| | | | | | SNORD115-48, ATP10A, | 15q11.2-q12, |
| | | | | | UBE3A, MKRN3, SNURF, | 15q12 |
| | | | | | SNORD64, NPAP1 | |

It can be seen from above results that the above-mentioned LOH region is located on chromosome 15, with a length of 12.28 M. FIG. 4 shows a diagram of the 12.8 M of LOH distribution on the chromosome, wherein the ellipse represents a LOH region. FIG. 5 shows an enlarged diagram of LOH distribution on chromosome 15.

4. Judging UPD

4.1 Principle Judgment

As such LOH region was not in accordance with the rules for judging consanguineous marriage and fragment deletion, the sample was judged as UPD.

4.2 Judging Pathogenic Risk

The above 12.28 M of LOH covers the imprinted gene which corresponds to Prader-Willi syndrome.

The sample is proved to have Prader-Willi syndrome later.

### EXAMPLE 3

A screening of a pathogenic UPD was performed on a sample by using the method of Example 1, wherein:

1. Obtaining Data

It was performed with reference to Example 1.

2. Screening for Sites

It was performed with reference to Example 1, and 22947 mutations meeting the pre-determined conditions were obtained.

3. Judging LOH

For the above obtained sites, a region was judged to be LOH if a product of an amount of contiguous homozygous sites and the coverage range thereof was greater than 200 Mbp, wherein the amount of contiguous homozygous sites was greater than or equal to 20, and the coverage range was greater than or equal to 3 Mbp.

According to the above rule, there were 2 LOH regions detected in the sample of this example, as shown in TABLE 3.

TABLE 3
LOH Regions

| | | | | Coverage | | |
|---|---|---|---|---|---|---|
| | | | | Range of | | |
| | | | Amount of | contiguous | | |
| | Start | End | homozygous | homozygous | Imprinted | Imprinted |
| Chromosome | Position | Position | mutations | sites(M) | gene | band |
| chr5 | 2748427 | 96350710 | 262 | 93.6 | ERAP2, | 5q15 |
| | | | | | RNU5D-1 | |
| chr5 | 167645888 | 180219304 | 109 | 12.57 | | |

It can be seen from above results that the above LOH regions are located on chromosome 5, with a length of 93.6 M and 12.57 M, respectively. FIG. 6 shows a diagram of both LOH regions distribution on the chromosome, wherein the ellipse represents LOH regions. FIG. 7 shows an enlarged diagram of 12.57 M of LOH according to FIG. 6.

Notes: CMA gene chip detection (chip type was CytoScan HD) was also done on the sample, and the tested results shows two LOH regions, i.e., chr5:2667631-99572420 and chr5:166974594-180520810, which are almost the same as those detected through the method of the present disclosure.

4. Judging UPD

4.1 Principle Judgment

As such LOH region was not in accordance with the rules for judging consanguineous marriage and fragment deletion, the sample was judged as UPD.

4.2 Pathogenic Risk Judgment

The above 93.6 M of LOH covered the imprinted genes ERAP2 and RNU5D-1. However, there are few studies related to them at present, so that they cannot be clearly identified as the cause of diseases, but can suggest relevant risks.

### EXAMPLE 4

A screening of a pathogenic UPD was performed by using a device as follows, the device comprises:

a module of data acquisition, configured for obtaining whole exome sequencing data;

a module of site screening, configured for screening for mutations under pre-determined conditions;

a module of LOH judgment, configured for performing LOH judgment according to the mutations obtained above; and a region is judged to be LOH when a product of an amount of contiguous homozygous sites and their coverage range is greater than a pre-set value; and

a module of UPD judgment, configured for performing UPD judgement according to the LOH judgment, wherein when an amount of chromosomes with LOH exceeds 2, a sample is judged as a consanguineous marriage; when there is a single copy of a region with LOH, a sample is judged as a fragment deletion; and other samples are judged as UPD when there are regions with LOH.

The above device run program according to the method of Example 1.

### EXAMPLE 5

A screening of a pathogenic UPD was performed by using the device of Example 4.

In this example, the whole exome gene sequencing obtained in routine examinations was analyzed, and five clinical samples were judged to be positive for UPD.

After a routine examination of the conventional whole exome gene sequencing, the above samples were analyzed with conventional methods and tested by MLPA. Among them, no clinically relevant and clear pathogenic variations were detected in 3 samples, but it was proved in methylation experiment that the above 5 samples were all PWS-AS, as shown in the following table.

TABLE 4
VERIFICATION RESULTS OF THE JUDGEMENT METHOD OF THE
PRESENT DISCLOSURE

| | | Verification | Results Reported |
|---|---|---|---|
| Sample | LOH Analysis | Results-Methylation | in a Conventional |
| number | Results | Level (%) | Method |
| NP19E1405 | 15q11q13 hmz | 4 | negative |
| NP19F0095 | chr15 hmz | 4 | negative |
| NP19E0517 | 15q14q21, 15q26 | 96 | chr15 UPD |
| | hmz | | |
| NP16S0255 | chr15 hmz | 98 | negative |
| NP16S0320 | 15q11q14 hmz | 86 | 15q11q14 del |

Note 1:
hmz is short for homozygous, indicating the region is homozygous, i.e., loss of heterozygosity (LOH).
Note 2:
For this region, the level of maternal-origin methylation is above 80% and the level of paternal-origin methylation is below 10%, so the methylation level in a normal people is about 45%. If the maternal-origin UPD occurs, the overall methylation level is above 80%, and the clinical manifestation is PWS (Prader-Willi syndrome); if the paternal-origin UPD occurs, the overall methylation level is below 10%, and the clinical manifestation is AS (Angelman syndrome).
Note 3:
The original reported results of sample NP16S0320 showed a large heterozygous region deletion of 15q11-q14, i.e., loss of one copy, which would also be indicated as LOH.

In the above samples, LOH results of sample NP19E1405 are shown in FIGS. 8-9, and verification results of methylation experiments are shown in FIG. 10. The results of sample NP19F0095 are shown in FIGS. 11-12, and the verification results of methylation experiment are shown in FIG. 13. The results of sample NP19E0517 are shown in FIGS. 14-15, and the verification results of methylation experiment are shown in FIG. 16. The results of sample NP16S0255 are shown in FIGS. 17-18, and the verification results of methylation experiment are shown in FIG. 19. The results of sample NP16S0320 are shown in FIGS. 20-21, and the verification results of methylation experiment are shown in FIG. 22.

### EXAMPLE 6

A screening of a pathogenic UPD was performed based on the whole exome sequencing data of 12444 samples, which were sent for screening pathogenic UPD. The screening was carried out according to the method in Example 1. 1018 samples were detected with LOH and 800 samples were remained apart from consanguineous marriage. After analysis, it was found that imprinted gene were covered in 142 samples, parts of which were proved to be consistent with the screening results at a coincidence rate of more than 95% after return visit.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. A method of screening a pathogenic uniparental disomy, comprising:
obtaining data: obtaining whole exome sequencing data;
screening for sites: screening and obtaining mutations under pre-determined conditions;
judging LOH: performing LOH judgement according to the mutations obtained in the step of screening for sites; and a region is judged to be LOH when a product of an amount of contiguous sites and a coverage range thereof is greater than a pre-set value; and
judging UPD: judging UPD according to the LOH judgement, wherein when an amount of chromosomes with LOH exceeds 2, a sample is judged as a consanguineous marriage; when there is a single copy of a region with LOH, a sample is judged as a fragment deletion; and other samples are judged as UPD when there are regions with LOH.

2. The method of screening a pathogenic uniparental disomy according to claim 1, wherein the mutations under the pre-determined conditions are screened and obtained through the following approaches:
screening for high-quality mutation sites: screening for high-quality mutation sites from the whole exome sequencing data;
removing Y chromosome mutations: removing Y chromosome mutations from the above mutation sites;
screening for point mutations: screening for point mutations from the mutations obtained in the step of removing Y chromosome mutations;
screening for allele frequency: screening for sites which are located in the point mutations in the previous step to obtain sites which have a population allele frequency of less than 0.7 in each race in a population database; and
screening for mutation frequency: removing sites which have a mutation frequency of heterozygous sites of higher than 70%, and removing sites which have a mutation frequency of homozygous sites of less than 85% from the sites which are located in point mutations in the previous step, thereby obtaining the mutations under predetermined conditions.

3. The method of screening a pathogenic uniparental disomy according to claim 2, wherein in the step of screening for high-quality mutation sites, the high-quality mutation sites are the mutation sites passed through a quality control of GATK-VQSR, and having a total coverage range of more than 40X and a mutation frequency of greater than 30%.

4. The method of screening a pathogenic uniparental disomy according to claim 2, wherein a step of excluding false positive sites is further included between the step of screening for allele frequency and the step of screening for mutation frequency, wherein the step of excluding false positive sites is performed according to the Hardy-Weinberg balance, by excluding false positive sites from a frequency database in a regional population to be evaluated.

5. The method of screening a pathogenic uniparental disomy according to claim 1, wherein the step of screening for high-quality mutation sites further includes a step of quality control, wherein the step of quality control is used to detect the amount of mutations obtained by the screening; when the amount of mutations is greater than or equal to 10,000, the step of quality control indicates PASS; when the amount of mutations is less than 10,000, the step of quality control indicates FAIL.

6. The method of screening a pathogenic uniparental disomy according to claim 1, wherein in the step of judging LOH, the amount of contiguous homozygous sites is greater than or equal to 20, and their coverage range is greater than or equal to 3 Mbp.

7. The method of screening a pathogenic uniparental disomy according to claim 6, wherein in the step of judging LOH, when the product of the amount of contiguous homozygous sites and their coverage range is greater than 200 Mbp, a region is judged to be LOH.

8. The method of screening a pathogenic uniparental disomy according to claim 1, wherein the step of judging UPD further includes a step of judging a pathogenic risk; in the step of judging a pathogenic risk, the LOH region which is judged to be UPD is further compared with imprinted genes; when the LOH region does not cover an imprinted gene or a corresponding band, a sample is indicated as a benign UPD; when the LOH region covers the imprinted gene or the corresponding band, a sample is indicated as being at risk of pathogenic UPD.

9. A method of preparing a device for screening a pathogenic uniparental disomy, comprising applying the method of claim 1 to screen a pathogenic uniparental disomy.

10. A device for screening a pathogenic uniparental disomy, comprising:
a module of data acquisition, configured for obtaining whole exome sequencing data;
a module of site screening, configured for screening for mutations under pre-determined conditions;
a module of LOH judgment, configured for performing LOH judgment according to the mutations obtained in the module of site screening, and a region is judged to be LOH when a product of an amount of contiguous homozygous sites and their coverage range is greater than a pre-set value; and
a module of UPD judgment, configured for performing UPD judgement according to the LOH judgment, wherein when an amount of chromosomes with LOH exceeds 2, a sample is judged as a consanguineous marriage; when there is a single copy of a region with LOH, a sample is judged as a fragment deletion; and other samples are judged as UPD when there are regions with LOH.

11. The device for screening a pathogenic uniparental disomy according to claim 10, wherein the mutations under pre-determined conditions are screened and obtained through the following approaches:
screening for high-quality mutation sites: screening for high-quality mutation sites from whole exome sequencing data;
removing Y chromosome mutations: removing Y chromosome mutations from the above mutation sites;
screening for point mutations: screening for point mutations from the mutations obtained in the step of removing Y chromosome mutations;
screening for allele frequency: screening for sites which are located in the point mutations in the previous step to obtain sites which have a population allele frequency of less than 0.7 in each race in a population database; and
screening for mutation frequency: removing sites which have a mutation frequency of heterozygous sites of higher than 70%, and removing sites which have a mutation frequency of homozygous sites of less than 85% from the sites which are located in point mutations in the previous step, thereby obtaining the mutations under predetermined conditions.

12. The device for screening a pathogenic uniparental disomy according to claim 11, wherein in the module of screening high-quality mutation sites, the high-quality mutation sites are the mutation sites passed through a quality control of GATK-VQSR, and having a total coverage range of more than 40X and a mutation frequency of greater than 30%.

13. The device for screening a pathogenic uniparental disomy according to claim 11, wherein a module of excluding false positive site is further included between the module of allele frequency screening and the module of mutation frequency screening, wherein the module of excluding false positive site is performed according to the Hardy-Weinberg balance, by excluding false positive sites from a frequency database in a regional population to be evaluated.

14. The device for screening a pathogenic uniparental disomy according to claim 10, wherein the module of sites screening further includes a quality control unit, wherein the quality control unit is used to detect the amount of mutations obtained by the screening; when the amount of mutations is greater than or equal to 10,000, the quality control unit indicates PASS; when the amount of mutations is less than 10,000, the quality control unit indicates FAIL.

15. The device for screening a pathogenic uniparental disomy according to claim 10, wherein in the module of LOH judgment, the amount of contiguous homozygous sites is greater than or equal to 20, and the coverage range is greater than or equal to 3 Mbp.

16. The device for screening a pathogenic uniparental disomy according to claim 15, wherein in the module of LOH judgment, when a product of the amount of contiguous homozygous sites and the coverage range thereof is greater than 200 Mbp, a region is judged to be LOH.

17. The device for screening a pathogenic uniparental disomy according to claim 10, wherein the module of UPD judgment further includes a unit of judging a pathogenic risk, wherein in the module of judging a pathogenic risk, the LOH region which is judged to be UPD is further compared with an imprinted gene; and
when the LOH region does not cover an imprinted gene or a corresponding band, this region is indicated as a benign UPD; when the LOH region covers the imprint gene or the corresponding band, the region is indicated as being at risk of pathogenic UPD.

18. A computer program product, comprising a computer readable storage medium storing a computer readable program code, the computer readable program code comprising an algorithm that when executed by a computer processor of a computing system implements the method according to claim 1.

19. A system comprising a processor configured to perform the method according to claim 1.
